(19) United States
(12) Patent Application Publication
(10) Pub. No.: US 2023/0320618 A1
(43) Pub. Date: Oct. 12, 2023

(54) METHODS, SYSTEMS, AND RELATED COMPUTER PROGRAM PRODUCTS FOR EVALUATING RESPIRATORY PATTERN

(71) Applicant: THE JOHNS HOPKINS UNIVERSITY, Baltimore, MD (US)

(72) Inventors: Mudiaga SOWHO, Baltimore, MD (US); Alexander BISANT, Ellicott City, MD (US); Daniel EVANS, Baltimore, MD (US)

(73) Assignee: THE JOHNS HOPKINS UNIVERSITY, Baltimore, MD (US)

(21) Appl. No.: 18/042,482

(22) PCT Filed: Aug. 25, 2021
(86) PCT No.: PCT/US2021/047501

§ 371 Date: Feb. 22, 2023

Related U.S. Application Data
(60) Provisional application No. 63/070,496, filed on Aug. 26, 2020.

(51) Int. Cl. A61B 5/08; A61B 5/00; A61B 5/0205

(52) U.S. Cl. CPC A61B 5/0816; A61B 5/4818; A61B 5/0205; A61B 5/7275

(57) ABSTRACT

Provided herein are methods of evaluating cardio-respiratory function, including detecting diseases, disorders, or conditions in test subjects involving the identification of respiratory timing patterns in respiratory audio signals. Related systems and computer program products are also provided.

### CROSS-REFERENCE TO RELATED APPLICATIONS

This application claims priority to, and the benefit of, U.S. Provisional Patent Application Ser. No. 63/070,496, filed Aug. 26, 2020, the disclosure of which is incorporated herein by reference.

### BACKGROUND

Changes to the respiratory pattern are known to be indicative of pulmonary pathology or stress to the respiratory system^{1, 2}. For instance, increase in the ratio of inspiratory time (Ti) to total respiratory time (Ttot) is a marker of upper airway obstruction seen in obstructive sleep apnea (OSA)^{1, 3}. Similarly abnormal increases in expiratory time (Te) is usually known to occur in disorders characterized by lower airway obstruction, as in asthma or COPD^{2, 6}. Since the respiratory system is connected to other organ systems, stress in those systems may change respiratory patterns as well. As an example, increase in respiratory rate (f_{R}) is commonly seen in worsening heart failure⁵. Precise characterization of respiratory patterns could thus provide a simple means to identify persons at risk for debilitating respiratory disease, and may also provide information regarding the severity of other disorders linked to the respiratory system. Current methods for characterizing respiratory patterns usually involve the use of devices and instrumentation that are cumbersome and could spread infection if used by different patients or involve multi-person contact.

Accordingly, there is a need for less intrusive and non-contact methods, and related aspects, for evaluating respiratory patterns to detect the presence and/or severity of diseases, disorders, or conditions.

### SUMMARY

The present disclosure relates, in certain aspects, to methods, systems, and computer readable media of use in evaluating respiratory pattern in subjects using respiratory audio signals originating from those subjects to detect the presence and/or severity of diseases, disorders, or conditions in those subjects.

In one aspect, the present disclosure provides a method of detecting a presence and/or severity of a disease, disorder, or condition in a test subject at least partially using a computer. The method includes (a) identifying, by the computer, one or more test respiratory timing patterns in one or more test respiratory audio signals originating from the test subject. The method also includes (b) calculating, by the computer, a degree of abnormality of the test respiratory timing patterns based on an algorithm that assigns an abnormality score, wherein the abnormality score correlates with the presence and/or severity of a given disease, disorder, or condition to thereby detect the presence and/or severity of the disease, disorder, or condition in the test subject, or (c) identifying, by the computer, one or more substantial matches between the test respiratory timing patterns and one or more reference respiratory timing patterns identified in one or more reference respiratory signals originating from one or more reference subjects, when the reference subjects are healthy or have the disease, disorder, or condition, wherein at least a subset of the reference respiratory timing patterns correlates with the presence and/or severity of the disease, disorder, or condition, in a given subject, thereby detecting the presence and/or severity of the disease, disorder, or condition in the test subject. In some embodiments, the method includes receiving the test respiratory audio signals originating from the test subject in substantially real-time. In certain embodiments, the method includes receiving a recording of the test respiratory audio signals originating from the test subject.

In certain embodiments, the method includes identifying the test respiratory timing patterns using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the test respiratory audio signals. In some embodiments, the method includes calculating the abnormality score of the test respiratory timing pattern using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the test respiratory audio signals. In some embodiments, the method includes identifying the reference respiratory timing patterns using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the reference respiratory signals. In certain embodiments of the method, the disease, disorder, or condition comprises one or more of asthma, chronic obstructive pulmonary disease (COPD), heart failure, sleep apnea, any respiratory disorder, any cardiovascular disorder, and any condition that modifies respiratory timing pattern. In certain embodiments of the method, the abnormality score of the test respiratory timing pattern is predictive of an impending asthmatic attack in the test subject. In certain embodiments of the method, the substantial matches between the test respiratory timing patterns and the reference respiratory timing patterns are predictive of an impending asthmatic attack in the test subject. In certain embodiments, the method includes administering one or more therapies (e.g., surgical intervention, therapeutic agents (e.g., pharmaceutical compositions, etc.), electromagnetic therapy (e.g., radiation, etc.), and the like) to the test subject to treat the disease, disorder, or condition. In some embodiments, the method includes repeating steps (a) and (b) or (c) at one or more different time points. In certain of these embodiments, the methods include administering one or more therapies to the test subject before, during, and/or after repeating steps (a) and (b) or (c) to treat the disease, disorder, or condition.

In another aspect, the present disclosure provides a system that includes at least one microphone, and at least one controller operably connected at least to the microphone, which controller comprises, or is capable of accessing, computer readable media comprising non-transitory computer-executable instructions which, when executed by at least one electronic processor perform at least: receiving one or more test respiratory audio signals originating from a test subject via the microphone; identifying one or more test respiratory timing patterns in the test respiratory audio signals; calculating an abnormality score of the test respiratory timing patterns, wherein the abnormality score correlates with the presence and/or severity of a given disease, disorder, or condition to thereby detect and/or characterize the severity of the disease, disorder, or condition in the test subject, or querying a database to identify one or more substantial matches between the test respiratory timing patterns and one or more reference respiratory timing patterns to detect the presence and/or severity of a disease, disorder, or condition in the test subject.

In some embodiments, the system comprises an operably connected database, which comprises one or more entries corresponding to one or more reference respiratory timing patterns identified in one or more reference respiratory signals originating from one or more reference subjects when the reference subjects are healthy or have the disease, disorder, or condition, wherein at least a subset of the reference respiratory timing patterns correlates with the presence and/or severity of the disease, disorder, or condition in a given subject to thereby detect and/or characterize the severity of the disease, disorder, or condition in the test subject. In certain embodiments of the system, a remote communication device (e.g., a desktop computer, a tablet computer, a mobile phone, and the like) comprises the microphone. In some embodiments of the system, the instructions perform identifying the test respiratory timing patterns using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the test respiratory audio signals. In some embodiments of the system, an algorithm calculates the abnormality score of the test respiratory timing pattern using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the test respiratory audio signals. In certain embodiments of the system, the reference respiratory timing patterns are identified using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the reference respiratory signals.

In some embodiments of the system, the disease, disorder, or condition comprises one or more of asthma, chronic obstructive pulmonary disease (COPD), heart failure, sleep apnea, any respiratory disorder, any cardiovascular disorder, and any condition that modifies respiratory timing pattern. In certain embodiments of the system, the abnormality score of the test respiratory timing pattern and/or the substantial matches between the test respiratory timing patterns and the reference respiratory timing patterns are predictive of an impending asthmatic attack in the test subject. In some embodiments of the system, the abnormality score of the test respiratory timing pattern and/or the entries of one or more members of the subset of the reference respiratory timing patterns are indexed to one or more therapies to treat the disease, disorder, or condition.

In another aspect, the present disclosure provides a computer readable media comprising non-transitory computer-executable instructions which, when executed by at least one electronic processor perform at least: identifying one or more test respiratory timing patterns in one or more test respiratory audio signals originating from a test subject; and calculating an abnormality score of the test respiratory timing patterns using an algorithm, wherein the abnormality score correlates with the presence and/or severity of a given disease, disorder, or condition to thereby detect the presence and/or severity of the disease, disorder, or condition in the test subject; or, identifying one or more substantial matches between the test respiratory timing patterns and one or more reference respiratory timing patterns identified in one or more reference respiratory audio signals originating from one or more reference subjects and when the reference subjects are healthy or have the disease, disorder, or condition, wherein at least a subset of the reference respiratory timing patterns correlates with presence and/or severity of the disease, disorder, or condition in a given subject to thereby detect the presence and/or severity of the disease, disorder, or condition in the test subject.

In some embodiments, the computer readable media comprises non-transitory computer-executable instructions which, when executed by at least one electronic processor further perform at least: receiving the test respiratory audio signals originating from the test subject in substantially real-time. In certain embodiments, the computer readable media comprises non-transitory computer-executable instructions which, when executed by the at least one electronic processor further perform at least: receiving a recording of the test respiratory audio signals originating from the test subject.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically depicts exemplary method steps according to some aspects disclosed herein.

FIG. 2 shows plots of sound level (dB(A)) and airflow (L/minute) over time.

FIG. 3 schematically depicts a nasal cannula disposed on the head of a subject.

FIG. 4 is a flow chart that schematically depicts exemplary method steps according to some aspects disclosed herein.

FIG. 5 is a flow chart that schematically depicts exemplary method steps according to some aspects disclosed herein.

FIG. 6 is a flow chart that schematically depicts exemplary method steps according to some aspects disclosed herein.

FIG. 7 schematically depicts exemplary method steps according to some aspects disclosed herein.

FIG. 8 show plots of data from a study according to one example disclosed herein. In particular, the plots show sound level and airflow channels from sleep study software, illustrating increasing sound level during snoring. T₀ and T_{end} are the times at the beginning and end of inspiration.

FIG. 9 shows plot of audio signal, sound level in decibel and transformed audio signal.

FIG. 10 shows plot of the correlation between the transformed audio signal vs the sound level signal in decibel (standard).

FIG. 11 shows plot of correlation between predicted and actual respiratory timing parameters (Te, Ttot). Predicted values were based on features of the respiratory audio signal and actual values were obtained from an airflow signal.

FIG. 12 shows Bland-Altman plot demonstrating prediction accuracy of method.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, systems, and component parts, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Classifier: As used herein, “classifier,” generally refers to algorithm computer code that receives, as input, test data and produces, as output, a classification of the input data as belonging to one or another class.

Indexed: As used herein, “indexed” refers to a first element (e.g., a respiratory timing pattern) linked to a second element (e.g., a given therapy).

Machine Learning Algorithm: As used herein, “machine learning algorithm,” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART—classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.”

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” For example, a subject can be an individual who has been diagnosed with having a respiratory disease, disorder, or condition, is going to receive a therapy for a respiratory disease, disorder, or condition, and/or has received at least one therapy for a respiratory disease, disorder, or condition.

Substantial Match: As used herein, “substantial match” means that at least a first value or element is at least approximately equal to at least a second value or element. The term “substantial match” also includes an exact match between the first value or element and the second value or element. In certain embodiments, for example, diseases, disorders, or conditions are detected when there is at least a substantial or approximate match between a given test respiratory timing pattern and a given reference respiratory timing pattern.

### DETAILED DESCRIPTION

Global Burden of Disease studies have estimated that about 300 million persons have asthma and about 251 million persons have chronic obstructive pulmonary disease (COPD) globally. The present disclosure provides methods, systems, and related software applications that have utility for the detection and monitoring of asthma and COPD as well as other chronic cardio-respiratory diseases, conditions, and other disorders that modify respiratory function. In certain aspects, the methods and related aspects utilize respiratory rate and/or inspiratory/expiratory timing acquired from audio recordings of a subject's breathing to detect and/or monitor respiratory pattern and function. In these embodiments, audio recording is typically obtained via a microphone in a mobile device (e.g. smartphone) or another type of communication device. In some embodiments, respiratory patterns captured as digital audio files are captured and then respiratory cycle parameters comprising an inspiratory time (Ti), an expiratory time (Te), a respiratory rate (f_{R}), a total respiratory cycle time (Ttot), and/or the like are determined. As described herein, audio recordings of breathing can be deconstructed into respiratory patterns. The methods and related aspects typically process breath sound characteristics to establish these respiratory patterns in order to: (1) monitor cardio-respiratory function, (2) increase diagnostic accuracy by determining the degree of respiratory pattern abnormality and/or matching with reference respiratory patterns of the given disorder compiled in a database, (3) create a unique respiratory digital fingerprint, and the like. Respiratory patterns are known to reflect underlying cardio-respiratory function, for example a) increased expiratory timing in asthma and COPD,^{2, 6} b) increased inspiratory timing in snoring/sleep apnea, and^{1, 3} c) increased respiratory rate in worsening heart failure⁵. Overall, methods and related aspects of the present disclosure provide an easy, precise and scalable way to detect and/or monitor highly prevalent debilitating chronic disorders in real-time as well as generate personalized respiratory patterns that improve precision medicine.

To further illustrate, exemplary utilities of the methods and related aspects disclosed herein include: (1) predicting an impending attack in asthmatic patients, prompting timely use of inhaler and vacating a location that has causative allergens unknown to the patient, (2) providing similar utility as in (1) in COPD patients at risk for an exacerbation, (3) providing therapy monitoring (e.g., effectiveness and efficacy of a given treatment can be monitored in personalized fashion), and (4) providing personalized respiratory patterns to help physicians and other healthcare providers to better understand their patients' conditions and to improve overall patient care.

Moreover, aspects of the present disclosure provide for the personalized characterization of respiratory function using non-contact mobile technology. The methods and related aspects disclosed herein can also be used to build global digital databases of respiratory patterns representative of known diseases and physiological phenotypes. In addition, the methods and related aspects of the present disclosure are readily scalable and have big data applications that allow interaction with artificial intelligence and machine learning techniques that advance knowledge, and the practice, of respiratory medicine.

Exemplary Methods

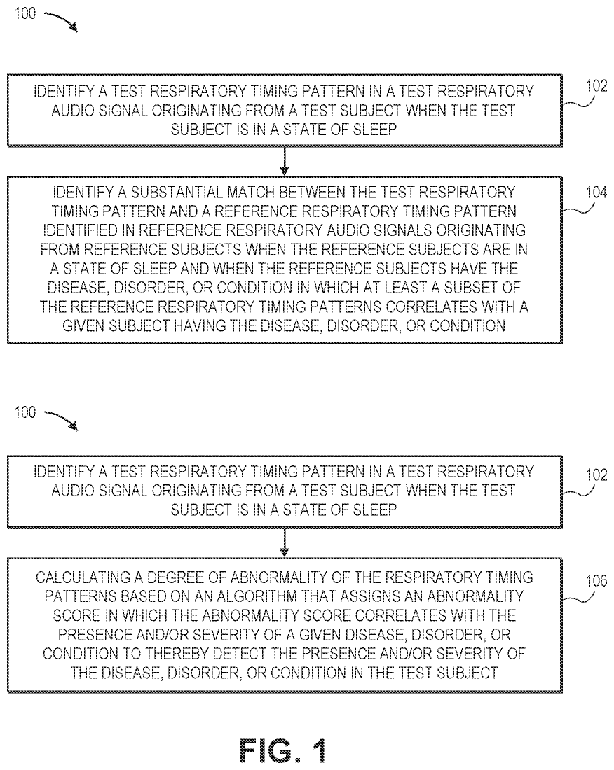

The present disclosure provides various methods of detecting a disease, disorder, or condition in a test subject. Typically, at least aspects of the methods disclosed herein are personalized or specific to a given test subject. To illustrate, FIG. 1 is a flow chart that schematically depicts exemplary method steps according to some aspects disclosed herein. As shown, method 100 includes identifying test respiratory timing patterns in one or more test respiratory audio signals (e.g., airflow signals associated with snoring) originating from the test subject (step 102). Typically, one or more of the steps of method 100 are computer implemented. Exemplary systems and computers are described further herein. In some embodiments, method 100 also includes identifying a substantial match between the test respiratory timing patterns and reference respiratory timing patterns (e.g., stored in a database or the like) (step 104). In some embodiments, method 100 also includes calculating a degree of abnormality of the respiratory timing patterns based on an algorithm that assigns an abnormality score in which the abnormality score correlates with the presence and/or severity of a given disease, disorder, or condition to thereby detect the presence and/or severity of the disease, disorder, or condition in the test subject (step 106), instead of step 104. In other embodiments, both steps 104 and 106 are also performed. The reference respiratory timing patterns are typically identified in reference respiratory signals (e.g., airflow signals associated with snoring) originating from reference subjects and when the reference subjects are healthy or have the disease, disorder, or condition under consideration. As also shown in step 104, at least a subset of the reference respiratory timing patterns correlates with the presence and/or severity of the disease, disorder, or condition in a given subject to thereby detect the disease, disorder, or condition in the test subject. In certain embodiments, one or more machine learning algorithms are applied to at least a portion of the reference respiratory timing patterns and/or reference respiratory audio signals (e.g., as a training data set) to generate a classifier of use in detecting diseases, disorders, or conditions in test subjects. In some embodiments, the methods and related aspects described herein are applied as a home based objective tracker/monitor of respiratory function in a given test subject.

In some embodiments, method 100 includes receiving the test respiratory audio signals originating from the test subject in substantially real-time (e.g., while the test subject is snoring). In other embodiments, method 100 includes receiving a recording of the test respiratory audio signals originating from the test subject (e.g., when the test subject was snoring).

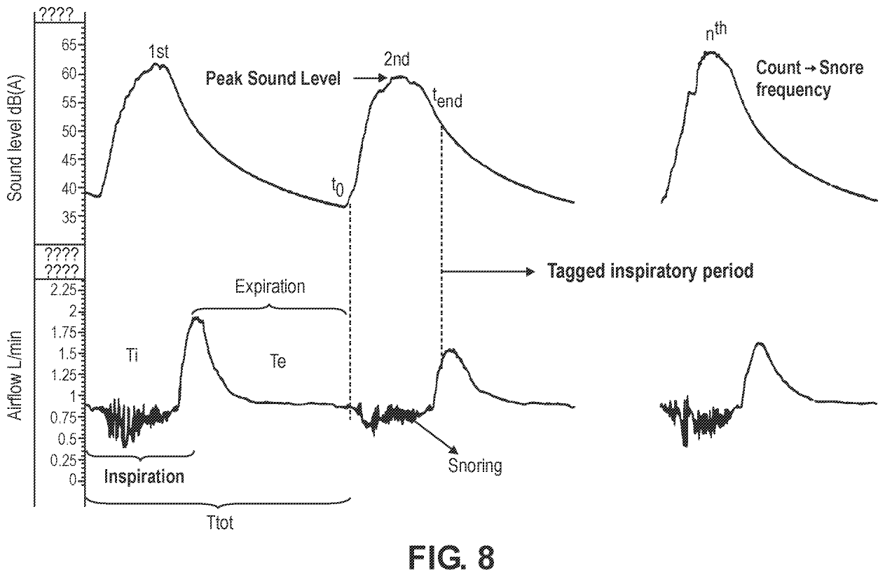

In certain embodiments, method 100 includes identifying the test respiratory timing patterns using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the test respiratory audio signals. In some embodiments, the method includes identifying the reference respiratory timing patterns using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the reference respiratory signals. For example, breath timing characteristics (inspiratory time (Ti), total respiratory time (Ttot) and the ratio of the both measures—Ti/Ttot) can be obtained from a simultaneously recorded airflow signal in certain embodiments (see, FIG. 8, discussed further herein). Changes in snoring sound level are associated with Ti/Ttot. Exemplary data showing this association is shown in Table 1 (Sowho et al)⁸. The association between breath timing and snoring sound is described further herein.

TABLE 1
Shows the association between with peak inspiratory sound in
decibels (dB(A)) and the severity of upper airway obstruction
represented by T_{I}/T_{TOT} (inspiratory duty cycle).

| Outcome: Peak inspiratory Sound | B | SE | p-value |
|---|---|---|---|
| Fixed Effects | | | |
| Intercept | 45.81 | 0.55 | <0.0001 |
| Tᵢ/T_{TOT} | 8.88 | 0.63 | <0.0001 |
| N1 | −6.46 | 0.23 | <0.0001 |
| N2 | −2.92 | 0.12 | <0.0001 |
| REM | −5.37 | 0.16 | <0.0001 |
| Non-Supine | −2.93 | 0.18 | <0.0001 |

| Random Effects | N |
|---|---|
| Subject ID | 77 |
| Observations | 15,597 |

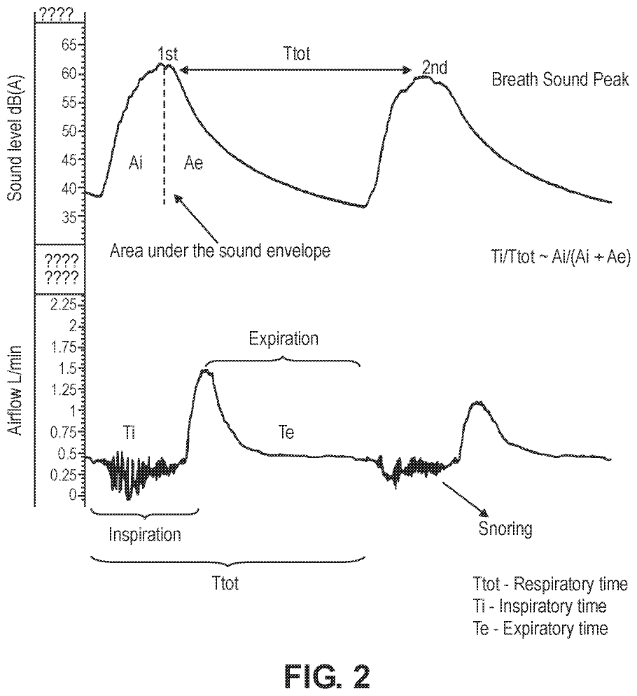

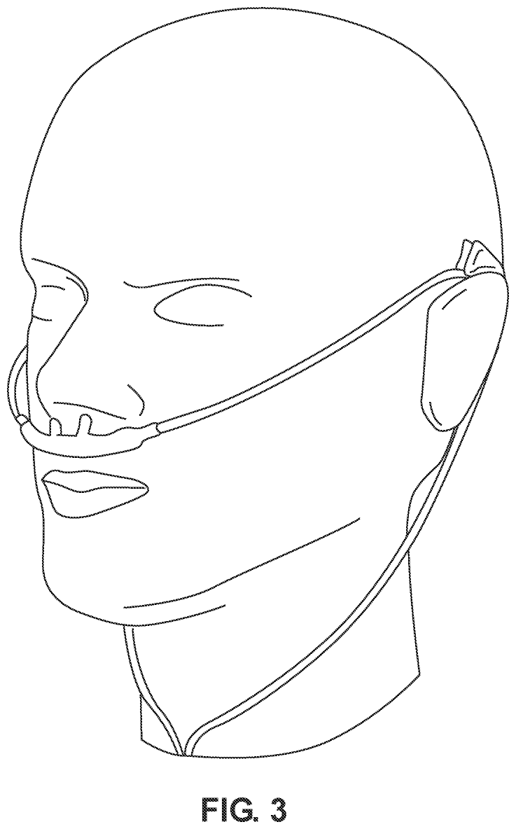

In some embodiments, the sound signal alone (without the airflow) can be used to derive the breath timing characteristics (see, e.g., FIG. 2). This is novel since respiratory timing parameters are typically derived from an airflow signal obtained from a nasal cannula, such as the one schematically depicted in FIG. 3.

The breath timing patterns derived from respiratory sounds have clinical utility for the prediction/diagnoses/detection and management of chronic respiratory disorders. Certain respiratory disorders have characteristic sounds and breathe timing patterns. For example, wheezing typically occurs in asthma and snoring with sleep apnea^{8, 9}. Although the sound alone may be indicative of the disorder, the timing feature improves accuracy by validating what the sound represents physiologically. In addition, changes in the breath timing pattern are informative and more sensitive for monitoring disease severity. Therefore, by using breath sounds as described herein, unique respiratory timing characteristics can be derived to enhance the prediction diagnoses/detection of underlying respiratory diseases, conditions, and disorders.

Accordingly, in certain embodiments of method 100, the disease, disorder, or condition comprises one or more of chronic obstructive pulmonary disease (COPD), heart failure, and sleep apnea. In certain embodiments of method 100, the substantial matches between the test respiratory timing patterns and the reference respiratory timing patterns are predictive of an impending asthmatic attack in the test subject. To further illustrate, in certain embodiments, method 100 also includes administering one or more therapies (e.g., surgical intervention, therapeutic agents (e.g., pharmaceutical compositions, etc.), electromagnetic therapy (e.g., radiation, etc.), and the like) to the test subject to treat the disease, disorder, or condition. In some embodiments, method 100 includes repeating steps (a) and (b) at one or more different time points (e.g., to monitor the disease, disorder, or condition in the test subject over time). In certain of these embodiments, method 100 includes administering one or more therapies to the test subject before, during, and/or after repeating steps (a) and (b) to treat the disease, disorder, or condition.

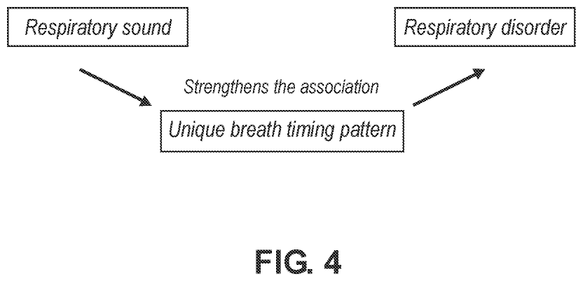

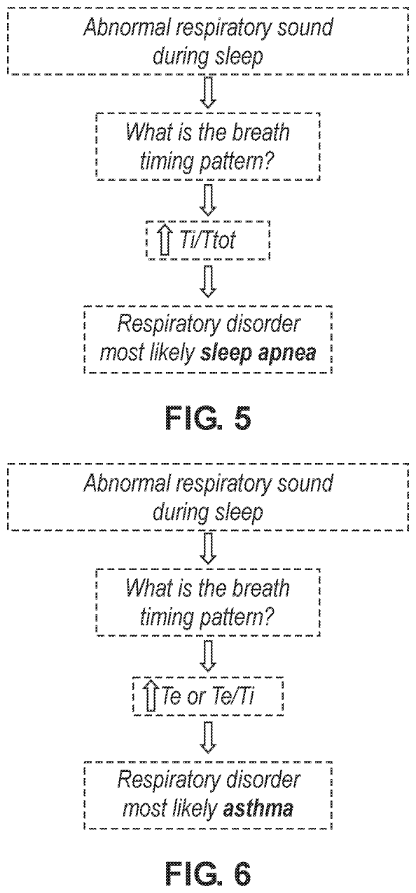

Additional exemplary aspects of the methods disclosed herein are depicted in FIGS. 4-6. FIG. 4, for example, schematically shows unique breath timing pattern be used to strength the association of respiratory sounds with particular respiratory disorders. FIG. 5 is a flow chart that schematically depicts exemplary method steps in which an abnormal respiratory sound from a test subject is detected during sleep, a breath timing pattern is determined, a Ti/Ttot ratio is calculated, and a sleep apnea respiratory disorder is detected using at least the calculated Ti/Ttot ratio. As a further illustration, FIG. 6 is a flow chart that schematically depicts exemplary method steps in which an abnormal respiratory sound from a test subject is detected during sleep, a breath timing pattern is determined, Te or a Te/Ti ratio is calculated, and an asthma respiratory disorder is detected using the calculated Te or a Te/Ti ratio.

Exemplary Systems and Computer Readable Media

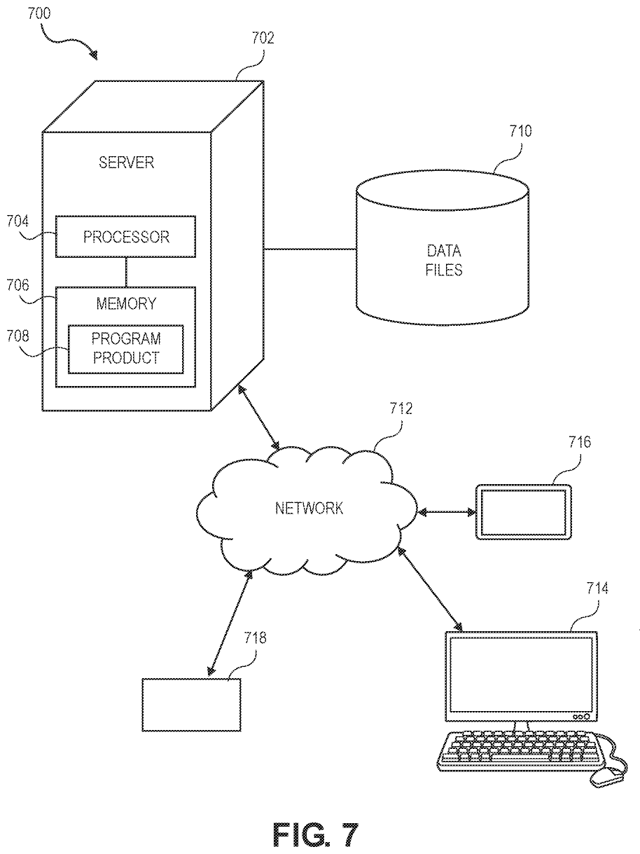

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 7 provides a schematic diagram of an exemplary system suitable for use with implementing at least aspects of the methods disclosed in this application. As shown, system 700 includes at least one controller or computer, e.g., server 702 (e.g., a search engine server), which includes processor 704 and memory, storage device, or memory component 706, and one or more other communication devices 714, 716, 718 (e.g., client-side computer terminals, telephones, tablets, laptops, other mobile devices, etc. (e.g., having microphones for receiving test respiratory audio signals, etc.)) positioned remote from and in communication with the remote server 702, through electronic communication network 712, such as the Internet or other internetwork. Communication devices 714, 716, 718 typically includes an electronic display (e.g., an internet enabled computer or the like) in communication with, e.g., server 702 computer over network 712 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 700 also includes program product 708 stored on a computer or machine readable medium, such as, for example, one or more of various types of memory, such as memory 706 of server 702, that is readable by the server 702, to facilitate, for example, a guided search application or other executable by one or more other communication devices, such as 714 (schematically shown as a desktop or personal computer). In some aspects, system 700 optionally also includes at least one database server, such as, for example, server 710 associated with an online website having data stored thereon (e.g., entries corresponding to more reference respiratory timing patterns, indexed therapies, etc.) searchable either directly or through search engine server 702. System 700 optionally also includes one or more other servers positioned remotely from server 702, each of which are optionally associated with one or more database servers 710 located remotely or located local to each of the other servers. The other servers can beneficially provide service to geographically remote users and enhance geographically distributed operations.

As understood by those of ordinary skill in the art, memory 706 of the server 702 optionally includes volatile and/or nonvolatile memory including, for example, RAM, ROM, and magnetic or optical disks, among others. It is also understood by those of ordinary skill in the art that although illustrated as a single server, the illustrated configuration of server 702 is given only by way of example and that other types of servers or computers configured according to various other methodologies or architectures can also be used. Server 702 shown schematically in FIG. 7, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 700. As also understood by those of ordinary skill in the art, other user communication devices 714, 716, 718 in these aspects, for example, can be a laptop, desktop, tablet, personal digital assistant (PDA), cell phone, server, or other types of computers. As known and understood by those of ordinary skill in the art, network 712 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 708 is optionally in the form of microcode, programs, cloud computing format, routines, and/or symbolic languages that provide one or more sets of ordered operations that control the functioning of the hardware and direct its operation. Program product 708, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 708 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 708 is optionally copied from the computer-readable medium to a hard disk or a similar intermediate storage medium. When program product 708, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes at least one respiratory time pattern or component thereof, and/or the like to be displayed (e.g., via communication devices 714, 716, 718 or the like) and/or receive information from other system components and/or from a system user (e.g., via communication device 714 or the like).

In some aspects, program product 708 includes non-transitory computer-executable instructions which, identifying one or more test respiratory timing patterns in one or more test respiratory audio signals originating from a test subject, and calculating a degree of abnormality of the respiratory timing patterns based on an algorithm that assigns an abnormality score in which the abnormality score correlates with the presence and/or severity of a given disease, disorder, or condition to thereby detect the presence and/or severity of the disease, disorder, or condition in the test subject, or identifying one or more substantial matches between the test respiratory timing patterns and one or more reference respiratory timing patterns identified in one or more reference respiratory audio signals originating from one or more reference subjects when the reference subjects are in a state of sleep and when the reference subjects have the disease, disorder, or condition, wherein at least a subset of the reference respiratory timing patterns correlates with a given subject having the disease, disorder, or condition to thereby detect the disease, disorder, or condition in the test subject.

System 700 also typically includes additional system components that are configured to perform various aspects of the methods described herein. In some of these aspects, one or more of these additional system components are positioned remote from and in communication with the remote server 702 through electronic communication network 712, whereas in other aspects, one or more of these additional system components are positioned local, and in communication with server 702 (i.e., in the absence of electronic communication network 712) or directly with, for example, desktop computer 714.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

### EXAMPLE

Association Between Peak Inspiratory Sound and Respiratory Timing

Sowho et al recently demonstrated a positive association between snoring sound and Ti/Ttot in a study were snoring was also shown to predict OSA accurately⁸. The investigation of the relationship between snoring sound and respiratory timing was conducted to confirm that sounds recorded were truly snoring. Since snoring is a marker of upper airway obstruction, breaths associated with snoring were expected to have an increased Ti/Ttot. The association of snoring sound intensity and Ti/Ttot, thus validated the sounds recorded as a) breath sounds, given that respiratory timing is a physiologic characteristic and as b) snoring sounds, since increased Ti/Ttot is a marker of upper airway obstruction. It is important to note, however, that the respiratory timing parameters for this project were obtained from an airflow signal (see FIG. 8) captured with a nasal cannula (see FIG. 3). Nonetheless the association between snoring (respiratory sound) and Ti/Ttot supports the notion that respiratory timing may be predicted directly from respiratory sound.

Novel Technology

Our technology thus extends the idea in the example by developing a means to predict respiratory timing parameters i.e. Ti, Te, f_{R}, Ttot, directly from breath sounds, and not via a nasal cannula or other device monitoring respiratory movements. To do this, we a) developed a reliable objective signal from raw audio recordings of breathing obtained with a smartphone and b) derived metrics from the objective audio signal useful for predicting respiratory timing (Ti, Te, f_{R} and Ttot).

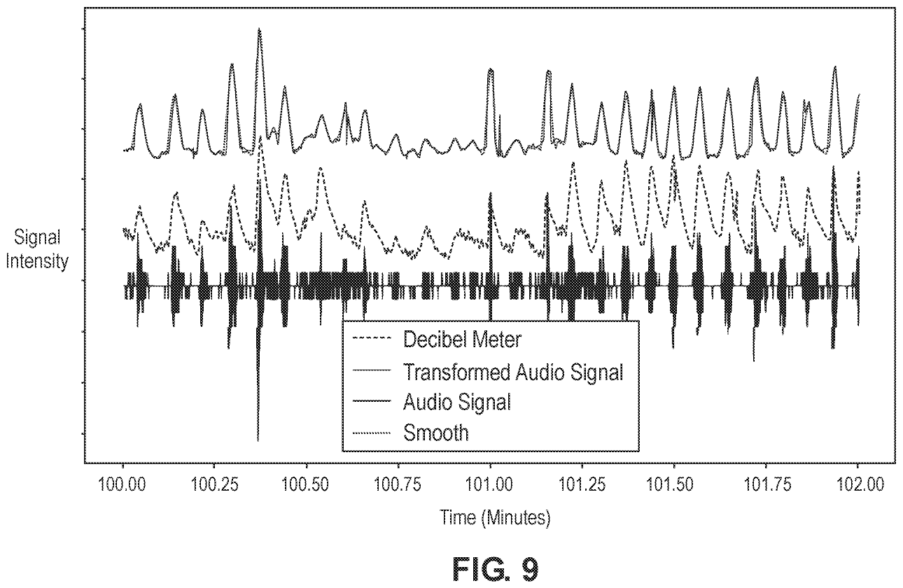

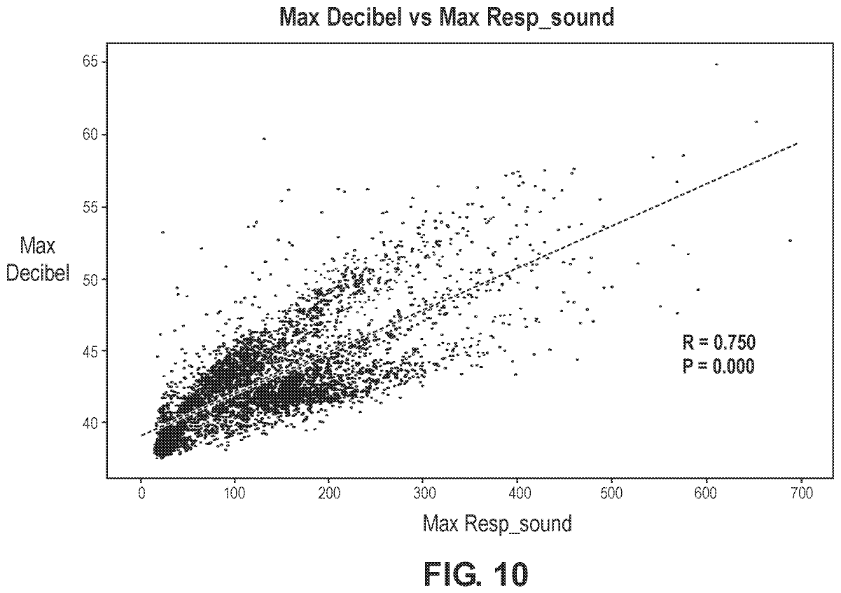

a) We recorded breath sounds from an index snorer with a commonly available mobile phone and a decibel meter simultaneously. The raw audio files were transformed into secondary signal (resp_sound), with objective metrics comparable to the decibel recording (see FIG. 9). FIG. 10 shows the correlation between the maximum decibel values and maximum resp_sound values per breath for the whole period of sleep. The values of the resp_sound do not represent a standard measure of sound loudness, but indicate a proportional increase in breath sound intensity relative to the breath sound level at baseline.

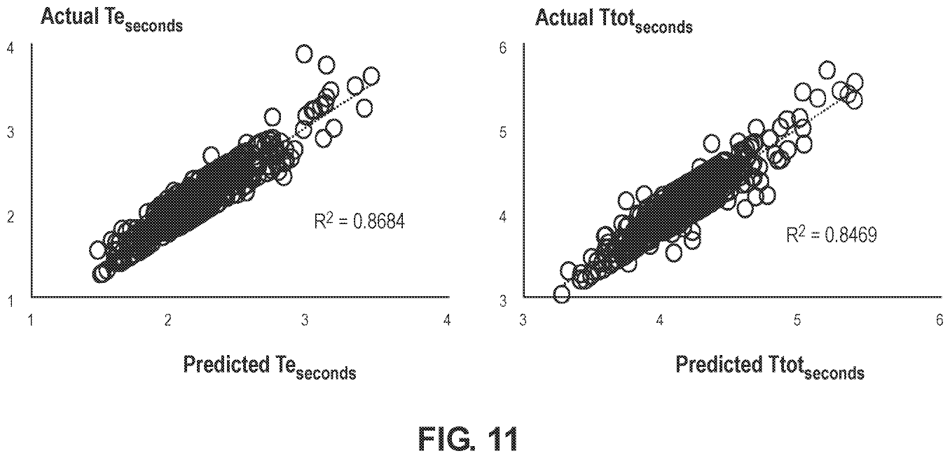

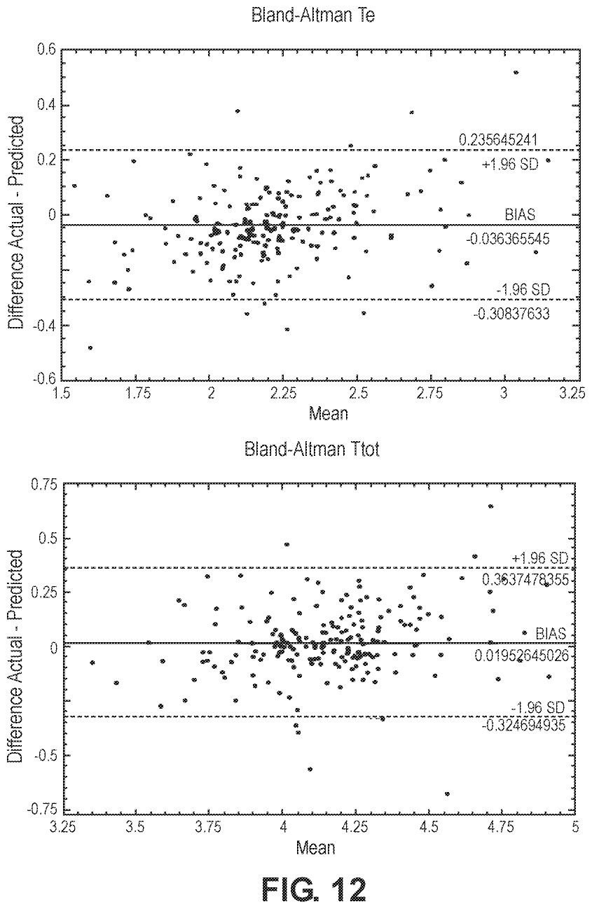

b) Some key features of resp_sound were combined into multivariable models and used to predict respiratory timing. Our data was split into training and validation sub-sets to test the accuracy of prediction. First, we show below the correlation between predicted and actual Te and Ttot in the training sub-sets (see FIG. 11). Second, Bland-Altman plots are also presented to demonstrate the prediction accuracy in the validation sub-sets (see FIG. 12). By deduction, Ti and f_{R} can be calculated from Te and Ttot (see Equation 1 and 2).

Tᵢ=Tₜₒₜ−Tₑ Equation 1:

f_{R}=1/Tₜₒₜ Equation 2:

Potential Implications

From the example discussed, objective measures of snoring severity constitute a strong predictor for concomitant OSA. Home-based assessments of snoring can thus facilitate OSA screening strategies in the community at large. Home-based assessment of snoring can be achieved using our novel non-contact technology that standardizes and streamlines the process of accurately characterize inspiratory snoring by extracting breath timing from the recorded snoring/respiratory sounds. In this example, for the purpose of OSA screening, the marker of disease presence and severity would be an increased Ti/Ttot^{1, 3}. Second, studies have shown that respiratory timing pattern during sleep is a reliable indicator of the presence of asthma, in which there is an abnormal increase in Te and Te/Ti². Routine assessment of these nocturnal breath timing parameters may thus inform medication use and adjustment of required dosage in asthmatic patients. Third, our custom algorithm and database of respiratory timing patterns will ultimately serve to promote precision medicine and customization of patient management. For instance, a slight decline in lung health may be discernable from changes in a patient's respiratory timing pattern, even before clinical symptoms emerge. It is also worth noting that given the nature of data capture and cloud storage with this technology, patient respiratory information would be made available to physicians and care givers real-time. Fourth, respiratory patterns have also been shown to indicate the severity of cardiovascular and metabolic disorders such as heart failure and diabetes^{5, 7}. Our technology provides a simple means to monitor disease progression and identify the patients at risk for readmission.

Finally, we are in a new era in which respiratory disease detection, monitoring and treatment is of paramount global public health importance. We can envisage utilizing our technology to characterize and establish the respiratory timing patterns of emerging pulmonary disorders, such as COVID-19, which will enhance our understanding of the disease. Our technology will also provide an easy to use non-contact means of monitoring the efficacy of new treatments, vaccines as well enable remote tracking of patient recovery from such respiratory diseases, even in low resource settings.

### REFERENCES

1. J Sanchis, J L Diez-Betoret, P Casan, J Milic-Emili. The pattern of resting breathing in patients with upper airway obstruction. Eur Respir J. 1990 May; 3(5):521-6.

2. Asai H, Furuya N, Ando T, Asai M, Yoshihara S, Ichimura T. Breathing patterns during sleep in stable asthmatic children. J Asthma. 1991; 28(4):265-72. doi: 10.3109/02770909109073383.

3. Schneider H, Krishnan V, Pichard L E, Patil S P, Smith P L, Schwartz A R. Inspiratory duty cycle responses to flow limitation predict nocturnal hypoventilation. Eur Respir J. 2009 May; 33(5):1068-76.

4. Mansour K F, Rowley J A, Badr M S. Noninvasive determination of upper airway resistance and flow limitation. J Appl Physiol (1985). 2004 November; 97(5):1840-8. Epub 2004 May 28.

5. Giovanni B Forleo, Luca Santini, Massimiliano Campoli, Mario Malavasi, Alberto Scaccia, Maurizio Menichelli, Umberto Riva, Filippo Lamberti, Giovanni Carreras, Serafino Orazi, Valentina Ribatti, Luigi Di Biase, Mariolina Lovecchio, Andrea Natale, Sergio Valsecchi, Francesco Romeo. Long-term monitoring of respiratory rate in patients with heart failure: the Multiparametric Heart Failure Evaluation in Implantable Cardioverter-Defibrillator Patients (MULTITUDE-HF) study. J Interv Card Electrophysiol. 2015 August; 43(2):135-44. doi: 10.1007/s10840-015-0007-3. Epub 2015 Apr. 28.

6. C B Cooper, G L Calligaro, M M Quinn, P Eshaghian, F Coskun, M Abrazado, E D Bateman, R I Raine. Determinants of dynamic hyperinflation during metronome-paced tachypnea in COPD and normal subjects. Respir Physiol Neurobiol. 2014 Jan. 1; 190:76-80. doi: 10.1016/j.resp.2013.08.002. Epub 2013 Aug. 27.

7. C J Weisbrod, P R Eastwood, G O'Driscoll, D J Green. Abnormal ventilatory responses to hypoxia in Type 2 diabetes. Diabet Med. 2005 May; 22(5):563-8. doi: 10.1111/j.1464-5491.2005.01458.x.

8. Mudiaga Sowho, Francis Sgambati, Michelle Guzman, Hartmut Schneider, Alan Schwartz. Snoring: a source of noise pollution and sleep apnea predictor. Sleep. 2020 Jun. 15; 43(6):zsz305. doi: 10.1093/sleep/zsz305.

[symbol]

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of detecting a presence and/or severity of a disease, disorder, or condition in a test subject at least partially using a computer, the method comprising:
(a) identifying, by the computer, one or more test respiratory timing patterns in one or more test respiratory audio signals originating from the test subject; and,
(b) calculating, by the computer, a degree of abnormality of the respiratory timing patterns based on an algorithm that assigns an abnormality score, wherein the abnormality score correlates with the presence and/or severity of a given disease, disorder, or condition to thereby detect the presence and/or severity of the disease, disorder, or condition in the test subject; or,
(c) identifying, by the computer, one or more substantial matches between the test respiratory timing patterns and one or more reference respiratory timing patterns identified in one or more reference respiratory signals originating from one or more reference subjects and when the reference subjects are healthy or have a disease, disorder, or condition, wherein at least a subset of the reference respiratory timing patterns correlates with the presence and/or severity of the disease, disorder, or condition, in a given subject thereby detecting the presence and/or severity of the disease, disorder, or condition in the test subject.

2. The method of claim 1, comprising receiving the test respiratory audio signals originating from the test subject in substantially real-time.

3. The method of claim 1, comprising receiving a recording of the test respiratory audio signals originating from the test subject.

4. The method of claim 1, comprising identifying the test respiratory timing patterns using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the test respiratory audio signals.

5. The method of claim 1, comprising calculating the abnormality score of the test respiratory timing pattern using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the test respiratory audio signals.

6. The method of claim 1, comprising identifying the reference respiratory timing patterns using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the reference respiratory signals.

7. The method of claim 1, wherein the disease, disorder, or condition comprises one or more of asthma, chronic obstructive pulmonary disease (COPD), heart failure, sleep apnea, any respiratory disorder, any cardiovascular disorder, and any condition that modifies respiratory timing pattern.

8. The method of claim 1, wherein the abnormality score for the test respiratory timing pattern and/or the substantial matches between the test respiratory timing patterns and the reference respiratory timing patterns are predictive of the presence and/or severity of asthma, chronic obstructive pulmonary disease (COPD), heart failure, sleep apnea, any respiratory disorder, any cardiovascular disorder, and any condition that modifies respiratory timing pattern, and/or wherein the abnormality score for the test respiratory timing pattern and/or the substantial matches between the test respiratory timing patterns and the reference respiratory timing patterns are predictive of an impending asthmatic attack in the test subject.

9. (canceled)

10. The method of claim 1, comprising administering one or more therapies to the test subject to treat the disease, disorder, or condition.

11. The method of claim 1, comprising repeating steps (a) and (b) or (c) at one or more different time points.

12. The method of claim 11, comprising administering one or more therapies to the test subject before, during, and/or after repeating steps (a) and (b) or (c) to treat the disease, disorder, or condition.

13. A system, comprising:
at least one microphone; and,
at least one controller operably connected at least to the microphone, which controller comprises, or is capable of accessing, computer readable media comprising non-transitory computer-executable instructions which, when executed by at least one electronic processor perform at least:
receiving one or more test respiratory audio signals originating from a test subject via the microphone;
identifying one or more test respiratory timing patterns in the test respiratory audio signals; and,
calculating an abnormality score of the test respiratory timing patterns, wherein the abnormality score correlates with the presence and/or severity of a given disease, disorder, or condition to thereby detect and/or characterize the severity of the disease, disorder, or condition in the test subject, or,
querying a database to identify one or more substantial matches between the test respiratory timing patterns and one or more reference respiratory timing patterns to detect the presence and/or severity of a disease, disorder, or condition in the test subject.

14. The system of claim 13, wherein the database comprises one or more entries corresponding to one or more reference respiratory timing patterns identified in one or more reference respiratory signals originating from one or more reference subjects when the reference subjects are healthy or have the disease, disorder, or condition, wherein at least a subset of the reference respiratory timing patterns correlates with the presence and/or severity of the disease, disorder, or condition in a given subject to thereby detect and/or characterize the severity of the disease, disorder, or condition in the test subject.

15. The system of claim 13, wherein a remote communication device comprises the microphone.

16. The system of claim 13, wherein:
the instructions perform identifying the test respiratory timing patterns using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the test respiratory audio signals;
the presence and/or severity of the disease, disorder, or condition are identified by an algorithm that calculates an abnormality score using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the test respiratory audio signals; or,
the presence and/or severity of the disease, disorder, or condition are identified using one or more respiratory cycle parameters comprising an inspiratory time (Ti), an expiratory time (Te), a respiratory rate (f_{R}), a total respiratory cycle time (Ttot), and/or a ratio of two or more of thereof, which respiratory cycle parameters are obtained from the reference respiratory signals.

17. (canceled)

18. (canceled)

19. The system of claim 13, wherein the disease, disorder, or condition comprises one or more of asthma, chronic obstructive pulmonary disease (COPD), heart failure, sleep apnea, any respiratory disorder, any cardiovascular disorder, and any condition that modifies respiratory timing pattern.

20. The system of claim 13, wherein:
the test respiratory timing patterns are deemed to achieve an abnormality score based on an algorithm and/or matches reference respiratory timing patterns, either of which are predictive of the presence and/or severity of the disease, disorder or condition in the test subject; or,
the test respiratory timing patterns are deemed to achieve an abnormality score and/or matches reference respiratory timing patterns, either of which are predictive of an impending asthmatic attack in the test subject.

21. (canceled)

22. The system of claim 13, wherein the abnormality score the test respiratory timing patterns and/or entries of one or more members of the subset of the reference respiratory timing patterns are indexed to one or more therapies to treat the disease, disorder, or condition.

23. A computer readable media comprising non-transitory computer-executable instructions which, when executed by at least one electronic processor perform at least:
identifying one or more test respiratory timing patterns in one or more test respiratory audio signals originating from a test subject; and,
calculating an abnormality score of the test respiratory timing patterns using an algorithm, wherein the abnormality score correlates with the presence and/or severity of a given disease, disorder, or condition to thereby detect the presence and/or severity of the disease, disorder, or condition in the test subject; or,
identifying one or more substantial matches between the test respiratory timing patterns and one or more reference respiratory timing patterns identified in one or more reference respiratory signals originating from one or more reference subjects and when the reference subjects are healthy or have a disease, disorder, or condition, wherein at least a subset of the reference respiratory timing patterns correlates with the presence and/or severity of the disease, disorder, or condition in a given subject to thereby detect the presence and/or severity of the disease, disorder, or condition in the test subject.

24. The computer readable media of claim 23, wherein the computer readable media comprising non-transitory computer-executable instructions which, when executed by the at least one electronic processor further perform at least:
receiving the test respiratory audio signals originating from the test subject in substantially real-time; and/or,
receiving a recording of the test respiratory audio signals originating from the test subject.

25. (canceled)
